(19) United States
(12) Patent Application Publication
(10) Pub. No.: US 2006/0276815 A1
(43) Pub. Date: Dec. 7, 2006

(54) Devices and methods for vessel harvesting

(75) Inventors: Richard Lotti, Saratoga, CA (US); Jeffery B. Alvarez, Brisbane, CA (US); James G. Whayne, Chapel Hill, NC (US); Kumar G. Jambunathan, North Smithfield, RI (US)

Correspondence Address: BOZICEVIC, FIELD & FRANCIS LLP, 1900 UNIVERSITY AVENUE, SUITE 200, EAST PALO ALTO, CA, 94303, US

(73) Assignee: Converge Medical, Inc.

(21) Appl. No.: 11/143,100

(22) Filed: Jun. 1, 2005

(51) Int. Cl. A61B 17/22

(52) U.S. Cl. 606/159

(57) ABSTRACT

Devices and methods for harvesting a body structure such as a vessel are provided. Embodiments include devices configured to harvest a vessel from a patient's body, wherein the device includes a mechanism for self-guiding the device over the vessel. Also provided are methods of using the subject devices to harvest a vessel from a patient's body.

### BACKGROUND OF THE INVENTION

Numerous surgical procedures involve accessing and harvesting a vessel from its native bed in a patient's body and transferring and using the harvested vessel at a another location in the patient' body. Procedures that involve vessel harvesting include coronary bypass surgery, femoral-tibial bypass, reverse or in situ femoral-popliteal procedures, and the like. For example, a vessel may be harvested for use in coronary artery bypass grafting (“CABG”), which is a surgical procedure to restore blood flow to ischemic heart muscle whose blood supply has been compromised by occlusion or stenosis of one or more of the coronary arteries. One method for performing CABG surgery involves harvesting a saphenous vein or other venous or arterial conduit from elsewhere in the body (e.g., radial arteries, cephalic veins), and connecting, via an anastomosis, this conduit from a viable arterial blood source, such as the aorta, to the coronary artery downstream of the blockage or narrowing. This allows the blood to flow through the graft vessel and bypass the narrowed or closed areas of the bypassed vessel.

A variety of different procedures have been developed for vessel harvesting. In the conventional or “open” method for harvesting a vessel, a long incision or series of incisions, that may stretch from a patient's groin to knee, knee to ankle or ankle to groin, in the case of saphenous vein harvesting, is made in the leg and the leg is splayed open to provide direct access to and visualization of the saphenous vein. Once the vein is removed, the incision or incisions are closed, e.g., by suturing or stapling. This conventional method is associated with high wound morbidity, extended post-operative recovery times, significant patient pain and results in disfiguring scars that may be cosmetically undesirable. An analogous open method may also be used to harvest other vessels from other locations in the body, e.g., the radial artery. However, in the case, e.g., of open radial artery harvesting, the trauma to the patient can be even greater, e.g., surgery can leave nerve damage and injury to the vessel.

In attempts to overcome the problems associated with conventional harvesting procedures, minimally invasive techniques have been developed in which one or more small incisions are made in the leg through which various surgical tools are inserted to harvest the vessel. These minimally invasive procedures have numerous advantages over the conventional procedures, including lower wound morbidity, shortened post-operative recovery periods, and do not produce the significant scars of the conventional method. However, because the leg is not completely opened in these procedures to provide visualization of the vessel of interest, typically an endoscope is required to view the saphenous vein inside the leg. Use of an endoscope requires the working space about the harvested vein to be large enough to accommodate the endoscopic equipment. Furthermore, multiple additional tools are used either serially or simultaneously to harvest the vessel, e.g., clip appliers, electrosurgical tools, tissue graspers, and the like. The use of a plurality of different tools may require more than one operator to complete the procedure and further increases the amount of working space needed about the vessel, especially when two or more tools are used at the same time.

An example of an endoscopic vessel harvesting system and technique is disclosed in U.S. Pat. No. 5,373,840 to Knighton. The scope housing has a lumen therethrough which is sized to accommodate the blood vessel being harvested and at least one tool, but more often is used to accommodate a plurality of tools such as for dissecting, gripping, coagulating, ligating and/or severing side branches and the vessel being harvested. The number of tools required and the need for more than one operator to perform the procedure are significant drawbacks. Furthermore, as the harvested vessel is received within the same instrument lumen through which the harvesting tools are delivered, the harvested vessel is subject to an increased risk of damage by the tools.

U.S. Pat. No. 5,772,576 to Knighton describes a device analogous to that disclosed in the '840 patent, but which includes a plurality of lumens for isolating the vessel from the tools in order to prevent the vessel from being damaged by the tools. In one embodiment, the body portion is sized large enough to include as many as five separate lumens: a viewing lumen, vessel lumen, irrigation lumen and one or two working lumens for the harvesting tools. In certain embodiments, the working and vessel lumens may be combined into a single lumen with a section for the vessel and a section for the tools, but in any event, the body portion is sized to include a plurality of different lumens. While the vessel may be isolated from the tools, the device of the '576 patent shares some of the same disadvantages of the device of the '840 patent. For example, multiple tools are required, and more than one operator is needed to complete the procedure. Furthermore, the various lumens of the body portion increase the size of the device.

U.S. Pat. No. 4,793,346 to Mindich discloses a vessel harvesting device which does not employ an endoscope. The '346 device includes a guide rod and an elongated tube having one or more sharp blades positioned at its distal end. The vessel is exposed through an incision and the guide rod is inserted into the vessel. The tube is guided over the vessel by the guide rod as it is passed over the vessel/guide rod structure. To sever the side branches, the tube is manually rotated as it is pushed along the vessel so that the blade(s) can sever side branches. The severed side branches can be cauterized by heating the blade(s). A disadvantage of this device is that it relies on manual translation of the device over the harvested vessel and tactile assessment by the operator in order to determine when an obstruction is encountered. Furthermore, while side branches are cut simultaneously by the blades as they are rotated, many of the side branches may not be adequately cauterized, resulting in significant blood loss by the patient. Still further, the sharp blade(s) may unintentionally damage tissue about the vessel and the vessel itself.

As vessel harvesting continues to be performed, there continues to be an interest in devices and methods for harvesting vessels which overcome the shortcomings of prior art devices and methods.

### SUMMARY OF THE INVENTION

Devices and methods for harvesting a body structure such as a blood vessel are provided. Embodiments include devices adapted to harvest a vessel from a patient's body, wherein the devices include a mechanism for self-guiding the device over the vessel.

Certain embodiments of the subject self-guided devices are adapted to process an obstruction encountered by the device such as tissue and/or a side branch. In one aspect, a subject device includes an inner tubular member surrounded by an outer tubular member, wherein the device includes an obstruction processing portion. For example, embodiments include inner and outer tubular members that are engageable at their distal ends by at least one groove and at least one tab, e.g., a plurality of grooves and tabs. The inner and/or outer member is rotatable to move the at least one tab and/or at least one groove to process an obstruction.

Also provided are methods for harvesting a body structure such as a vessel from a patient's body. Embodiments of the subject methods include making an incision in a patient's body, transecting a vessel adjacent the incision to provide a free end of the vessel, inserting a device over the free end of the vessel, the device including a mechanism for self-guiding the device over the vessel, and actuating the mechanism to advance the device over the vessel, whereby the device is self-guided over the vessel.

Also provided are systems and kits for use in practicing the subject methods.

### BRIEF DESCRIPTIONS OF THE DRAWINGS

The figures shown herein are not necessarily drawn to scale, with some components and features being exaggerated for clarity.

FIGS. 1A-Dare schematic illustrations of self-guiding mechanism use with the harvesting devices of the present invention including a first windlass drive mechanism, another windlass drive mechanism, a gear reduction system and a worm gear system, respectively.

FIG. 2 shows an exemplary embodiment of a self-guided vessel harvesting device according to the subject invention.

FIG. 3 shows another exemplary embodiment of a self-guided vessel harvesting system of the present invention.

FIG. 4A shows an exemplary embodiment of side branch processing portion that includes an inner tubular member surrounded by an outer tubular member and which may be employed with the harvesting devices of the present invention.

FIG. 4B shows an exploded view of the side branch processing portion of FIG. 4A.

FIG. 4C shows a cross-sectional view of the device of FIG. 4A positioned over a vessel having a side branch connected thereto.

FIG. 5 shows a section of a saphenous vein to be removed from a leg of a patient.

FIG. 6 shows a self-guided vessel harvesting device of the subject invention in use to harvest the saphenous vein of FIG. 5.

### DETAILED DESCRIPTION OF THE INVENTION

As summarized above, subject invention includes devices and methods for removing a body structure, such as a hollow anatomical structure, from a patient's body, where the devices and methods of the subject invention are particularly useful for harvesting a blood vessel such as a saphenous vein, radial artery, cephalic vein, and the like, e.g., for use in a CABG procedure. The subject invention is described primarily with respect to harvesting a blood vessel such as a saphenous vein or the like from a patient's body, where such description is not intended to limit the scope of the invention in any way. It will be apparent to those of skill in the art that the subject invention may be employed to remove a variety of body structures, e.g., tendons, and the like.

Certain embodiments include devices that have tubular body portions or tubular structures that are linearly translatable over a vessel to be harvested, where translation of the device is self-guiding or self-tracking, requiring minimal to no visual or tactile assessment or evaluation by the operator. In particular, the self-guided devices are adapted to translate over the harvested vessel, which translation is interrupted by the presence of tissue obstructions such as side-branches and tributaries. Certain embodiments of the device may be adapted to “process” such obstructions in order to continue or further translate the device over the harvested vessel. As used herein, “processing” is meant to refer to cauterizing the obstruction and/or separating the obstruction from the vessel being harvested, e.g., cauterizing a side branch and/or dividing a side branch to free it from the vessel being harvested. As described in greater detail below, certain embodiments of the subject devices include obstruction processing portions.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All patents and publications mentioned herein are incorporated herein by reference to disclose and describe the methods and/or materials in connection with which the patents and publications are cited. The citation of any patent or publication is for its disclosure prior to the filing date and should not be construed as an admission that the present invention is not entitled to antedate such patent or publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

As described above, embodiments of the subject invention include devices that are adapted to be self-guided over an anatomical body structure, such as a blood vessel or the like, to be harvested from a patient's body. The devices of the subject invention include a mechanism (“the mechanism” or the “self-guiding mechanism”) for self-guiding the device over an anatomical structure such as a blood vessel to be harvested from the patient's body. In certain embodiments, the self-guiding mechanism is a linear translation mechanism. The self-guided devices of the subject invention provide a number of advantages. For example, a subject self-guided vessel harvesting device eliminates the need to manually push the device along the vessel to be harvested and thereby obviates the need for a viewing scope such as an endoscope or other visualization system to guide the device. Furthermore, a subject self-guided device does not rely on tactile assessments by the operator in order to determine when an obstruction has been encountered.

Embodiments of the subject devices include a tubular body portion having a lumen configured to receive a blood vessel to be harvested. As the device is self-guided over the vessel, the vessel is captured within the lumen of the device for easy removal of the vessel from the patient's body.

The vessel-receiving tubular body portion of the device is connected to the above-described mechanism so that activation of the mechanism linearly translates the tubular body portion of the device over a vessel in a self-guiding manner. The self-guiding mechanism is adapted to automatically halt further linear translation of the device over a vessel in at least the forward (distal) direction once an obstruction such as a side branch is encountered by the device. In this manner, use of the device does not rely on the tactile observation and subsequent reaction by the operator to stop the translation once an obstruction is encountered. Embodiments may also include an operator indicator such as a visual and/or audio alert (not shown) for notifying the operator when translation is halted due to an obstruction.

The mechanism for self-guiding the device over a vessel may be any suitable mechanism. In certain embodiments, the mechanism is a linear translation mechanism for selectively self-guiding and linearly translating the device (or portion thereof) over the vessel. The mechanism may be adapted to translate the device unidirectionally (i.e., forward (distally) along the vessel being harvested) or bidirectionally (i.e., both forward and backward (proximally)).

As described in greater detail below, once an obstruction is encountered, it may then be processed, e.g., by the device itself in certain embodiments, after which the mechanism may again be activated for further advancement of the device over the vessel. For example, the portion of the vessel that will be removed from the patient must be separated from tributary vessels that branch off the saphenous vein (i.e., side branches) along its length.

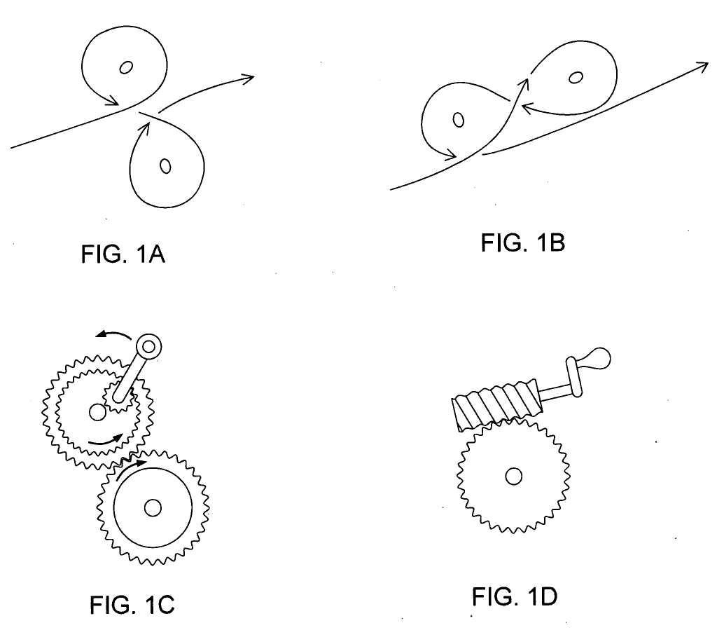

In certain embodiments, the self-guiding mechanism may be a geared mechanism or assembly. The gear assembly may be operated or driven by any suitable means including, but not limited to, an electric, pneumatic, or hydraulic motor with a user control (linear or proportional), by a ratchet lever, by a knob, by a reel, by a trigger with a ratchet, and the like, but in any event is configured to advance the tubular body portion over a vessel in a self-guiding manner. Various gear systems are known in the art and may be employed. For example, in certain embodiments a windlass drive mechanism (FIGS. 1A and 1B) may be used that may be hand-cranked or motor driven or electrically or pneumatically operated. Other configurations are possible as well, for example embodiments may include a gear reduction system (FIG. 1B) or a worm gear arrangement (FIG. 1C), and the like.

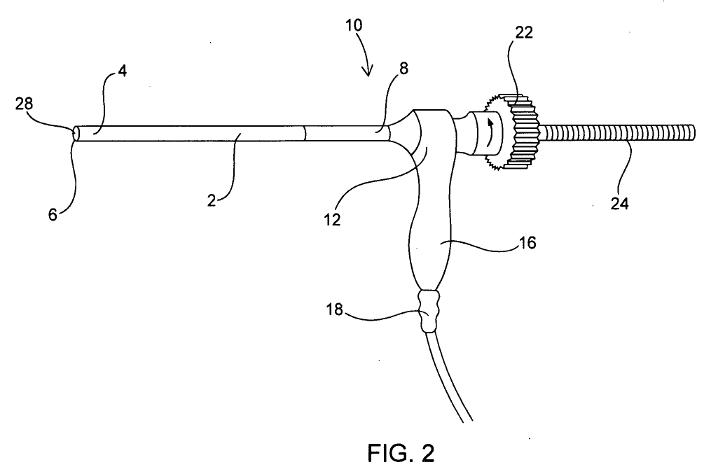

FIG. 2 shows an exemplary embodiment of a self-guidable vessel harvesting device 10 according to the subject invention. Device 10 includes elongated tubular body portion or shaft 2 having a distal or leading end 4 terminating at distal edge 6 and a proximal end 8 and defining a lumen 18 therebetween which is adapted to accommodate therein a vessel 24 to be harvested. Lumen 18 extends proximally through a handle portion 12 to allow proximal advancement and exiting of a harvested portion of vessel 24.

Handle 12 is preferably ergonomically configured for the operator to manually hold and control the device. Handle 12 includes griping portion 16 and a connector 18 which allows for coupling with a power source (not shown) and current for electrocautery, a fluid source (not shown) for flushing the harvesting site and/or a source of vacuum (not shown) for smoke and fluid evacuation. In the embodiment of FIG. 1, handle 12 houses a self-guiding mechanism (not shown) as described above for guiding the device over a vessel to be harvested. Handle 12 also includes an activation means, such as a trigger mechanism or the like (not shown) for selectively activating the guiding mechanism. The activation means may be located elsewhere on the device or at a remote location, e.g., a foot pedal, etc. Handle 12 may also include one or more additional control means for controlling aspects of the device other than the self-guiding, linear translation of the device. For example, as described in greater detail below, in certain embodiments, shaft 2 may be selectively rotatable to process an obstruction. In such embodiments, handle 12 (or another portion of device 10) may include means to control the selective rotation of shaft 2. For example, in this particular embodiment handle 12 includes proximally-positioned control knob 22 rotatably mounted on handle 12 and coupled to tubular body portion 2 for selectively controlling the rotational movement of the tubular body portion.

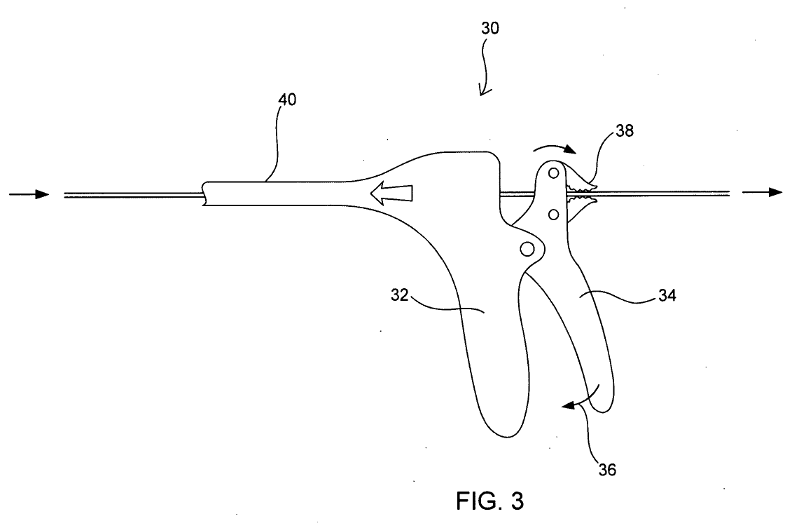

FIG. 3 shows another exemplary embodiment of a subject device 30 that employs a ratcheting system to linearly translate the device over a vessel. Device 30 includes a handle 32 having trigger 34 which, when actuated in the direction of arrow 36, causes opposable jaws 38 to open and linear translation of tubular body portion 40 a defined distance. Upon release of the trigger, the jaws close, thereby maintaining the tubular body portion in a fixed position. The present invention may also use springs or biasing members to reduce the amount of force needed to push the trigger (or knob, lever, etc.). As mentioned above, the trigger system may be used to translate the device unidirectionally or bidirectionally.

The shaft or tubular body portion of the subject devices may be fabricated from any suitable biocompatible material. In certain embodiments, at least a portion of the tubular body portion may include electrically conductive material. A portion of the tubular body portion, e.g., the inner walls of the tubular body portion, may be coated or layered with a material to facilitate ease of movement over a vessel being harvested, e.g., a non-stick material and/or lubricious material. For example, the lumen of tubular body portion may include a polymeric material to increase its lubricity and to minimize the potential for trauma as it moves over a vessel to be harvested. The inner walls of the tubular body portion may be coated or layered with an anti-thrombogenic material. Various polymers, and the like, that may be used for these purposes are known to those of skill in the art. For example, coatings which may be adapted for use with the subject invention are described, e.g., in U.S. Pat. No. 6,706,025, the disclosure of which is herein incorporated by reference, and elsewhere.

Tubular body portion may have any suitable dimensions, where in certain embodiments the tubular member has a small outer diameter dimension, e.g., slightly larger than the vessel being harvested. Tubular body portion may have a constant cross-sectional outer diameter along its entire length or may have a gradually reduced cross-sectional area along at least a portion of its length. For example, the distal end of the shaft may have a cross-sectional area that gradually reduces towards a distal edge. Such embodiments may minimize resistance to forward motion of tubular body portion and may also facilitate separation of tissue from the vessel while tubular body portion is advanced over the vessel to be harvested. In certain embodiments, the tubular body portion may include a plurality of tubular members, e.g., coaxially arranged. For example, a tubular body portion may include a first smaller diameter tubular member and a second larger diameter tubular member.

As described in greater detail below, certain embodiments of the subject devices are adapted to process an obstruction and include an inner tubular member disposed within an outer tubular member, wherein the inner tubular member is rotatable with respect to the outer tubular member and/or the outer tubular member is rotatable with respect to the inner tubular member.

In certain embodiments, the first and second tubular members may be telescopically mounted with respect to each other and operation of the self-guiding mechanism causes the smaller member to be telescoped from the larger member. For example, the smaller tubular member may telescopically interconnect with the larger tubular member and may be moved in and out of the larger tubular member to adjust the overall length of the device to approximate the desired length of vessel to be harvested.

As described above, during vessel harvesting procedures, obstructions may be encountered. For example, a side branch encountered by the device which must be severed from the vessel and permanently closed before the device continues advancement over the vessel. Before a side branch is cut, it is typically ligated by applying a clip, cutting it away from the main vessel and then permanently closing it, e.g., by suturing, clipping, or cauterizing. Accordingly, the subject invention may be adapted for use with various tools for processing tissue or a side branch. Embodiments may be adapted to accommodate one or more tools in the same lumen of the device in which the vessel is accommodated, or the device may include one or more additional lumens for accommodating the vessel in one lumen and one or more tools in one or more other lumens. Alternatively, a working space may be provided or created exterior to the device for accommodating various tools.

Using separate tools to process a side branch requires separate steps, which may increase the time required for the surgery. Accordingly, certain device embodiments may include an obstruction processing portion integrated into the device such that the device itself is configured to process an obstruction such as a side branch. In this regard, such embodiments may be characterized as all-in-one devices in that means for processing (e.g., ligating and/or clipping, and/or cutting and/or cauterizing, etc) an obstruction is integrated into the device itself, where certain embodiments are adapted to provide controlled cutting and coagulation of a side branch in one step. These all-in-one devices obviate the need for separate tools and additional space for accommodating the tools.

Certain embodiments may include a processing portion that includes a cutting member, e.g., attached to the device, e.g., at the distal end of the device, for severing a side branch. The cutting member may be in the form of one or more blades. A blade may have one cutting edge, for unidirectional cutting, or two cutting edges for bidirectional cutting. The one or more blades may be rotatable with respect to the vessel, e.g., about an axis thereof, to divide a side branch connected to the vessel. For example, one or more blades may be attached to the distal end of a tubular body portion and the blade-carrying tubular body portion may be rotatable. In certain embodiments, one or more blades may be attached to an annular member connected to, and rotatable about, the tubular body portion. Rotation of the tubular body portion or annular member thereof may be accomplished by any suitable mechanism, a knob, button, lever, switch, or the like, e.g., positioned on handle 12 of device 10 of FIG. 2, such as knob 22. The processing portion of a device may be adapted to apply electrocautery energy to a side branch. For example, one or more of the attached blades may serve as an electrode for applying monopolor or bipolar energy to a side branch.

In certain embodiments, a device that includes an integrated obstruction processing portion may include a shaft that has an inner tubular member disposed within an outer tubular in a coaxial relationship, wherein the inner and outer members, or portions thereof, provide the obstruction processing portion of the device and as such are adapted to provide opposable grasping and/or cutting and/or electrode surfaces to grasp, and/or cut and/or cauterize an obstruction such as a side branch.

More specifically, in such embodiments both the inner and outer tubular members include a proximal end and a distal end with a lumen therebetween. The inner and outer tubular members are engageable at their distal ends by way of at least one radially extendable tab and at least one corresponding tab-accepting groove, e.g., a plurality of tabs and corresponding grooves, wherein the tabs/grooves are used to process and obstruction. For example, the distal end of the inner tubular member may include at least one radially extending tab, e.g., multiple radially extending tabs, and the distal end of the outer member may be slotted to produce multiple fingers spaced apart by at least one tab-accepting groove, e.g., multiple tab-accepting grooves. The inner tubular member is positionable within the lumen of the outer member such that the radially extending tabs of the inner tubular member are received in the grooves of the outer tubular member in a manner that provides sufficient space so that at least one of the tubular members may rotate relative to the other tubular member. The subject devices that include inner and outer member are described primarily with respect to a plurality of radially extending tabs and a plurality of corresponding tab-accepting grooves, where such is for exemplary purposes only and is in no way intended to limit the scope of the invention as it is to be understood that any number of tabs/grooves may be used, including one tab and one groove.

The some or all of the inner and/or outer members may include electrically conductive material. For example, at least a portion of the radially extending tabs of the inner member may include an electrically conductive material and/or at least a portion of the grooves of the outer member may include electrically conductive material. In any event, the device is adapted so that current may be passed between electrically conductive portions of the inner and outer tubular members to process a side branch positioned adjacent a tab/groove portion. Ultrasonic, radio frequency (RF), laser technology, and the like, may also be employed instead of electrosurgical technology, and the inner and outer members suitably adapted therefore.

Specifically, in certain embodiments the distal end of the vessel harvesting devices of the subject invention may be rotatably movable between a first position which may be characterized as a tissue-accepting position in which a space for receiving an obstruction such as a side branch or the like is provided between a surface of a radially extending tab of an inner member and a surface of the corresponding groove of an outer member, and a second position which may be characterized as a tissue-processing position in which the obstruction is secured or held between the surface of the radially extending tab and the surface of the groove. In the tissue processing position, a side branch may be held, e.g., compressed, between a surface of a tab and surface of a groove to hold it in a fixed position and/or to cut the side branch using compression force and/or electrical energy may be applied to the side branch to divide the side branch and/or to cauterize it. Compression applied to the vessel may provide improved vessel sealing. Movement between the first and second positions may be accomplished by rotational movement of the inner and/or outer member, e.g., by a control means associated with the device such as knob 22 of handle 12.

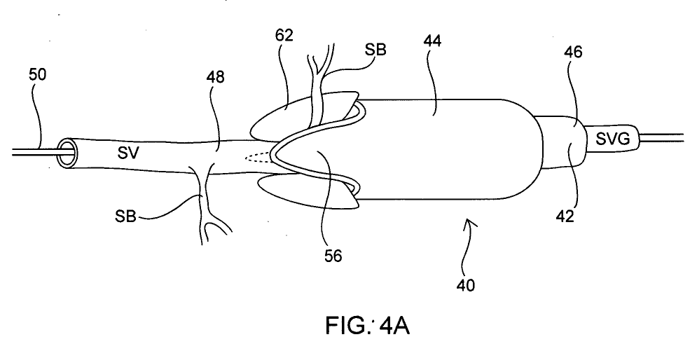

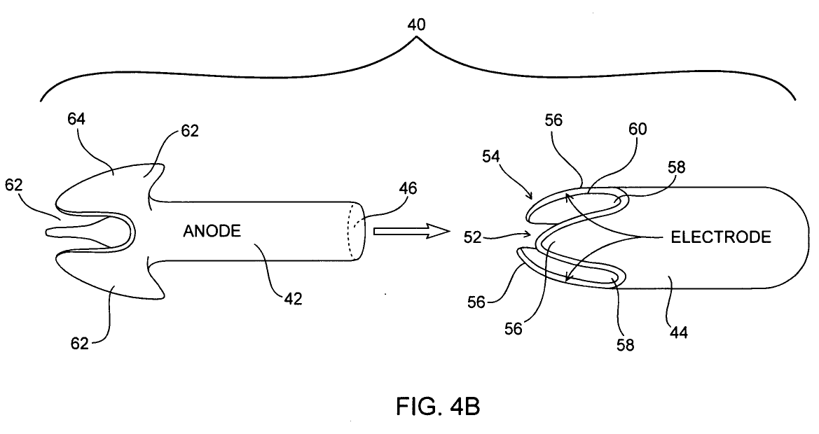

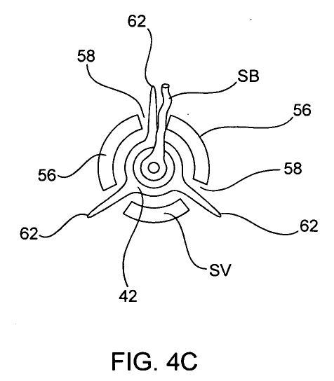

FIGS. 4A-4C illustrate various views of an obstruction processing distal portion 40 of an exemplary embodiment of a subject tissue harvesting device. Portion 40 includes an inner member 42 positioned within outer member 40. Both members have tubular configurations and are coaxially aligned. Outer lumen 44 defines a lumen 52 for accommodating at least a portion of inner member 42. Inner member 42 defines a lumen 46 dimensioned to accommodate a vessel to be harvested, herein shown as saphenous vein SV having side branches SB connected thereto. Outer member 44 has an inner diameter which need only be large enough to accommodate the inner member 42, and thus may be smaller than prior art devices that require a size large enough to accommodate various tools such as endoscopes, and the like. At least one of the members may move freely relative to the other member, i.e., to enable outer member 44 to be selectively rotated relative to inner member 42 and/or to enable inner member 42 to be selectively rotated relative to outer member 44.

Distal end 54 of outer tubular member 44 includes multiple finger-like projections 56 extending longitudinally from the distal end of the tubular member and which are spaced apart to provide grooves 58 between the fingers for receiving a side branch or other obstructive structure. Fingers 56 and grooves 58 may have any suitable shape and size. Fingers 56 may all have the same size (length and/or width dimension) and/or shape or may include fingers, all or some of which may differ in size (length and/or width dimension) and/or shape. The width of a finger may be substantially constant along its length such as to form a rectangle or the like or, in certain embodiments, the width of a finger may vary so that a finger may have a smoothly tapered shape, i.e., a gradually reduced width dimension, e.g., to form a V-shape or triangularly-shaped finger, or the like. For example, one or more of the fingers may be formed as a tapered projection longitudinally extending from tubular member 44. Fingers 56 are spaced-apart a distance to accommodate a corresponding tab 62 of inner tubular member 42 and/or an obstruction such as a side branch SB that may be encountered during vessel harvesting.

The pattern of spacing between the fingers, and thus the pattern of spacing of the grooves formed by the fingers, may be a regular pattern (constant or uniform) or irregular pattern. That is, all or some of the fingers may be regularly space-apart from each other such that the spacing between at least some of the fingers may be the same or the spacing may be irregular such that the spacing between at least some of the fingers may differ from the spacing between one or more other fingers. Stated otherwise, some or all of the grooves of an outer member distal end may have the same dimensions and/or shape or some or all of the grooves may have different dimensions and/or shape.

Fingers 56 having a tapered shape are well suited for causing an obstruction such as a side branch contacted by a finger during linear translation of the device over the vessel, and more particularly contacted by the distal or leading edge 60 of a finger, to slip into a groove 58 of the device. In this manner, an obstruction encountered by the distal edge 60 is guided into a space between the fingers.

The distal end of inner member 42 includes a plurality of radial extensions 62 in the form of tabs, protrusions, fins or blades or the like, each having a distal or leading edge 64. Grooves 66 may also be provided between adjacent extensions 62. As shown in FIG. 4A, extensions 62 are employed together with the grooves/fingers of the outer member to process an obstruction such as a side branch SB connected to a vessel to be harvested and which has been encountered by the device. Specifically, an extension 62 is configured to fit within a groove 58 in the manner shown in FIG. 4A. As such, the number of extensions 62 typically, but are not required to, corresponds to the number of fingers 56 or grooves 58. In this particular embodiment, three tabs and three fingers are shown, however embodiments may include more or less than three tabs-finger pairs.

Extensions 62 may have the same size and shape or some or all may be of different sizes and shapes, e.g., some may be narrower or wider than others. They may have any suitable shape such as, but not limited to, the shark-fin shape shown in FIGS. 4A-4C. Extensions preferably have a shape and size, such as the fin design of the illustrated embodiment, to guide a side branch into a groove 58 of outer member 44.

At least a portion of the leading edge 60 of outer member 44 and/or a portion of the surfaces of extensions 62 of inner member 42 may be formed from an electrically conductive material to function as an electrode for cauterizing and/or cutting tissue. Certain embodiments of the subject invention may be adapted for monopolar electrosurgery, in which only one of the inner or outer tubular member is provided with electrodes. Other embodiments may be configured for bipolar electrosurgery in which two electrodes, one of which may be in the form of or be a part of the outer member and the other may be in the form of or a part of the inner member, with the two electrodes being electrically isolated from each other.

A current source (not shown) which may be selectively activated by the user may be coupled to the device for applying and regulating a voltage to the one or more electrodes of the device, e.g., for applying and regulating a voltage between the electrodes of the inner and outer members in certain bipolar configurations. The current source may be an electrosurgical unit capable of providing bipolar energy, as noted above. The energy may be delivered through electrically insulated wires to one or more of the electrodes. The application of electrical current to the distal tip of the device may be controlled manually by the operator of the apparatus, e.g., by a switch or other mechanism. For example, a switch may be located on a handle of the device or at a remote location (e.g., a foot switch). In certain embodiments, a system may be configured to apply electrical energy automatically and continuously to one or more of the electrodes, or may be applied automatically at selected times when a side branch enters a groove of the device, e.g., upon rotation of the inner and/or outer member a predetermined distance.

As mentioned above, in certain embodiments, at least one of the inner and outer members is rotatable with respect to the other for processing an obstruction therebetween. For example, an obstruction such as a side branch extending from the side of a vessel may be processed by positioning the obstruction between a surface of an inner member extension 62 and a surface of an outer member finger 56. Rotation of at least one of the tubular members such as by the handle or by independent rotation of a remote actuation element, brings a surface of a tab and surface of a finger together or in sufficient proximity to process the side branch therebetween, as shown in the cross sectional view of FIG. 4C. As such, embodiments of the devices of the subject invention are capable of serially or simultaneously applying both (1) electrical energy to an obstruction such as a side branch of a vessel, and (2) mechanical pressure to an obstruction.

Also provided by the subject invention are systems that include a self-guidable vessel harvesting device according to the subject invention.

In certain instances it may be desirable to provide support to the vessel being harvested. As such, a system of the subject invention may include a vessel support or tension member insertable into the lumen of a vessel being harvested. The support member is of suitable dimensions to fit inside a vessel to be harvested. Any suitable structure capable of supporting a vessel being harvested may be used. For example, a support member may be in the form of an elongated cable, wire, or the like. In certain embodiments, the support member may also provide illumination of the working area, e.g., the vessel and surrounding area thereof such as side branches, surrounding tissue, etc. In certain embodiments, a support member may be a light-emitting fiber optic cable or bundle thereof connected to a source of light. FIGS. 4A and 6 show embodiments that include a vessel support member 50. For example, the system of FIG. 6 includes support member 50 in the form of a fiber optic cable connected to a light box.

One or more tools or components for use with the device may also be included in a system. For example, a system may include one or more of: an electrosurgical generator, source of fluid for flushing the site (e.g., saline contained in a reservoir), a vacuum source for evacuating electrocautery smoke, a support member, and the like. In those embodiments which include a support member, the support member may be in the form of a light-emitting fiber optic cable or bundle thereof. In systems that employ a light emitting support member such as a fiber optic cable or bundle support member or the like, the system may also include a light source to be used with a light emitting support member. A system may also include tools for processing an obstruction such as clamps, clips, cutting members, and the like.

As noted above, the subject devices may be used for a variety of different procedures, including but not limited to vessel harvesting procedures. Embodiments of the subject methods for harvesting a vessel from a patient's body generally include making an incision in a patient's body, transecting a vessel adjacent the incision to provide a free end of the vessel, inserting a device over said free end of the vessel wherein the device includes a mechanism for self-guiding the device over the vessel, and actuating the mechanism to advance the device over the vessel, whereby the device is self-guided over the vessel.

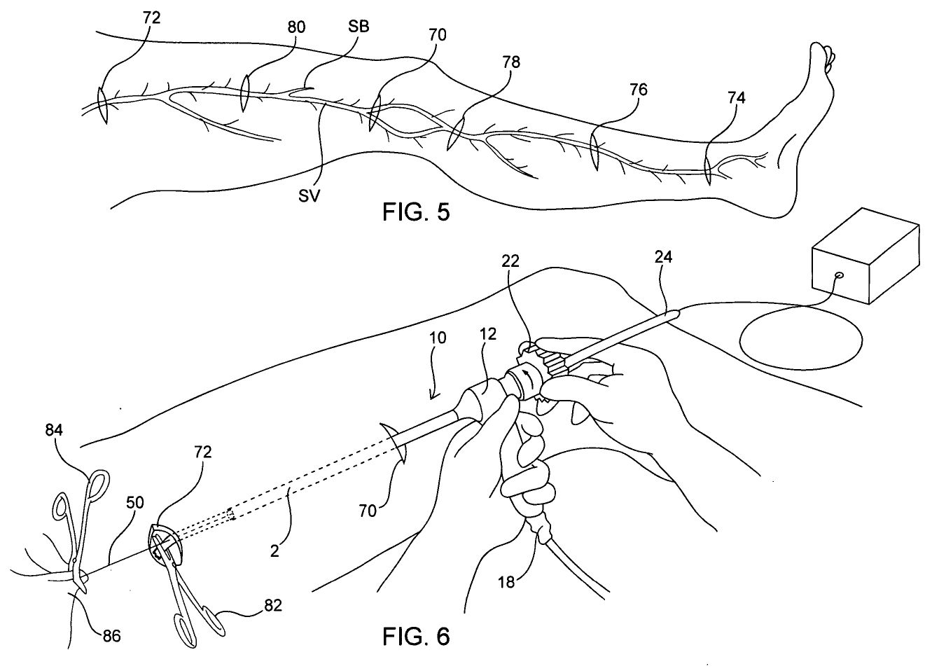

FIGS. 5 and 6 illustrate an exemplary method of the subject invention employing self-guided device 10 of FIG. 2 in an exemplary vessel harvesting procedure, herein described with reference to removing a section of a saphenous vein. Accordingly, embodiments of the subject invention include self-guiding a subject device over a vessel to be harvested.

FIG. 5 shows a section of a saphenous vein SV to be removed from a leg of a patient. To remove a section of saphenous vein SV, one or more incisions 70, 72, 74, 76, 78 and 80 are made over a portion of the saphenous vein. For example, the incisions may involve a knee cutdown and a groin cutdown over portions of the saphenous vein to provide an access point to the vein and point of introduction for a self-guided vessel harvesting device of the subject invention. The distance between the two incisions representing the length of vein section to be harvested. One or more additional incision may be also be made, e.g., an additional incision 74 may be made at the ankle area, e.g., and at other locations in between, to harvest the entire length of the saphenous vein. An exemplary vessel harvesting procedure is described herein primarily with respect to two incisions, a first (proximal) device entry incision 70 and a second (distal) incision 72. It will be apparent from the description that the subject methods may be adapted to include a greater or lesser number of incisions.

At first incision 70, the vein is transected to provide a free end. The free end is inserted into the vessel receiving lumen of the device. The free end may first be dissected a length, e.g., under direct vision, and then inserted into the lumen of the device. At second incision 72, the vein may be ligated, e.g., using a clamp 82 as shown, or the like.

Certain embodiments may include supporting the vein and/or illuminating the vein during the procedure. For example, embodiments may include using tension member 50 to support the vein during the procedure, which may be a light emitting tension member such as a fiber optic cable or the like. The tension member 50 may be inserted through the lumen of the device, inserted into the free end of the vein and passed through the length of the vein section and out the second incision 72, where it may be anchored, e.g., to surgical drapes 86 or the like, such as by using a clamp 84 or the like. The tension member 50 is not necessary to advance the self-guiding device over the vein section, however it may be employed in certain instances, e.g., to provide a light source so that side braches may be viewed through the skin.

Once a subject device is positioned over the free proximal end of the vessel, the device and more particularly the tubular body portion of the device, is self-guided and linearly translated over the vein in a manner described above. More particularly, the device does not require the operator to manually push the device along the vein. Instead, actuation of a mechanism of the device self-tracks and linearly translates the device over the vein. Accordingly, the operator grasps the proximal end of the device, e.g., a handle at the proximal end of the device, and selectively actuates the self-guiding translation mechanism, where actuation may be by way of a button, switch, lever, etc., as described above. In certain embodiments, the operator actuates a trigger of the handle, which advances the tubular member of the device a distance over the vein section in a self-guiding manner. In those embodiments that include telescopically mounted first and second tubular members, advancement of the device over the vessel may include telescopically moving the first tubular member in and out of the second tubular member. Because the device is self-guided over a vessel being harvested, a scope or other viewing device is not required to harvest a vessel using a subject device.

Translation of the device is automatically halted when the device encounters an obstruction such as a side branch. Once a side branch is encountered, it must be processed in order for the device to continue linear translation in a forward or distal direction. Processing an obstruction may be accomplished in a number of different ways. In certain embodiments, various separate tools for processing a side branch may be advanced to the side branch and used, e.g., through the same or different lumen of the device or advanced over or under the device.

In certain embodiments, the device may include an integrated obstruction processing portion and the method may include processing an obstruction using the integrated obstruction processing portion of the device. For example, the tubular body portion of the device may include inner and outer tubular members forming a distal obstruction processing portion of radially extending tabs and corresponding tab-accepting grooves, as described above. Once an obstruction is positioned in a groove of the device, the device may be used to at least grasp an obstruction for processing. The inner member and/or outer member may be rotated to at least grasp the obstruction between a tab and a finger (see, e.g., FIG. 4C), where a compression force may be applied to compress the obstruction between the tab and finger. For example, the proximal end of the device may be held and controlled by the operator, while the distal end may be used to process an obstruction. An operator may actuate the device from the proximal end of the device to cause at least a portion of the device to rotate to trap the obstruction between a tab and a finger of the device, e.g., by selectively rotating a knob or the like positioned on a handle at the proximal end of the device, or the like. In certain embodiments, in addition to grasping the obstruction, the aforementioned may cause pressure to be applied to the obstruction. Monopolar or bipolar electrosurgical current may be applied through the instrument by selectively activating a current source (e.g., by way of a foot peddle or the like) to the obstruction, e.g., using an inner member electrode and/or outer member electrode. The electrical energy may be used for cauterization and/or to divide the obstruction. As noted above, ultrasonic energy, radio frequency (RF), laser energy, etc., may also be used.

Certain embodiments may include positioning an obstruction encountered by the device in a suitable position for processing. Positioning an obstruction for processing may be automatic in certain embodiments in that no or little action by the user is required to position the obstruction for processing, e.g., the configuration of the device itself may guide an obstruction into a groove of a device upon contact where it may be processed. For example, the distal portion of the device such as the distal edge (e.g., the distal edge of the fingers and/or the tabs), may guide an obstruction into position in a groove so that it may be processed.

Once an encountered obstruction is processed, the device may again continue along the vessel until the desired length of vessel has been dissected, which length may be defined by second incision 72. Accordingly, once an obstruction is processed, the device is further moved along the vessel by actuation of the self-guiding translation mechanism, processing obstructions encountered along the way to separate the vessel from surrounding tissue and side branches, in a manner analogous to that described above.

At the end of the procedure (when the device has been advanced a sufficient length over the vessel), a suitable length of vessel is conveniently confined within the lumen of the device for easy removal. The vein is transected at the distal end, if not already done. The desired length of saphenous vein may then be extracted via the entry incision by removing the device through the entry incision with the vein contained therein. The device may be removed by simply pulling the device in a rearward (proximal) direction and out through the entry incision manually, or by actuating the translation mechanism to cause linear translation of the device in a proximal or reverse direction as that which was used to remove the vessel from its native bed.

Finally, kits are also provided. The subject kits may include a self-guided vessel harvesting device of the subject invention. In certain embodiments, a kit may include a plurality of different, interchangeable tubular body portions. For example, the tubular body portions may differ in size and/or shape from one another, e.g., different lengths and/or inner diameters and/or outer diameters, to provide the physician convenience and security of having a device with the correct size for a particular patient.

A kit may include other tools or components for use with the device, e.g., one or more of: an electrosurgical generator, a fluid source for flushing the site, a vacuum source for evacuating electrocautery smoke, a support member which may be in the form of a light-emitting fiber optic cable or bundle thereof, a light source to be used with a light emitting support member, etc. A kit may also include tools for processing an obstruction such as a clamp, clip, cutting member, and the like. Some or all of the components of a kit, e.g., at least the patient contacting components, may be provided sterile in packaging sufficient to ensure sterile integrity.

The subject kits may also include written instructions for using the device of a kit. Instructions of a kit may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g. via the Internet, are provided. An example of this embodiment is a kit that includes a web address where the instructions can be viewed and/or from which the instructions can be downloaded. As with the instructions, this means for obtaining the instructions is recorded on a suitable substrate.

In certain embodiments of the subject kits, the components of the kit are packaged in a kit containment element to make a single, easily handled unit, where the kit containment element, e.g., box or analogous structure, may or may not be an airtight container.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A device adapted to harvest vessel from a patient's body, said device comprising a mechanism for self-guiding the device over said vessel.

2. The device of claim 1, wherein said mechanism is a linear translation mechanism.

3. The device of claim 2, wherein said linear translation mechanism comprises a ratchet system.

4. The device of claim 2, wherein said linear translation mechanism comprises a windlass system.

5. The device of claim 2, wherein said linear translation mechanism comprises gear reduction.

6. The device of claim 2, wherein said linear translation mechanism comprises a worm gear.

7. The device of claim 2, wherein said linear translation mechanism comprises a motor.

8. The device of claim 2, wherein said device comprises a handle and said linear translation mechanism is associated with said handle.

9. The device of claim 2, wherein linear translation mechanism is adapted to automatically halt linear translation of said device when an obstruction is contacted.

10. The device of claim 1, wherein said device comprises a proximal end and a distal end and said distal end comprises a processing portion for processing an obstruction contacted by said distal end.

11. The device of claim 10, wherein said distal end of said device is configured to perform at least one of: ligate, cut, electrocauterize, grasp and compress.

12. The device of claim 1, wherein said device comprises a tubular body portion having a proximal end, a distal end and a lumen therebetween.

13. The device of claim 10, wherein said lumen is configured to receive said vessel.

14. The device of claim 13, wherein said tubular body portion comprises an inner tubular member and an outer tubular member.

15. The device of claim 14, wherein at least one of said inner tubular member and said outer tubular member is selectively rotated with respect to the other.

16. The device of claim 15, wherein said inner member comprises at least one radially extending tab and said outer member comprises at least one groove for receiving said at least one radially extending tab.

17. The device of claim 14, wherein at least a portion of said inner tubular member and at least a portion of said outer tubular member comprise an electrically conductive material.

18. The device of claim 14, wherein said inner tubular member and said outer tubular member are telescopically interconnected.

19. A system for harvesting a vessel from a patient's body, said system comprising:
a device adapted to harvest vessel from a patient's body, said device comprising a mechanism for self-guiding the device over said vessel; and
an electrosurgical generator.

20. The system of claim 19, wherein said mechanism is a linear translation system.

21. The system of claim 20, further comprising a vessel support member.

22. The system of claim 21, wherein said vessel support member is a light emitting vessel support member.

23. The system of claim 22, wherein said vessel support member is a light-emitting fiber optic cable or bundle thereof.

24. A method for harvesting a vessel from a patient's body, said method comprising:
making an incision in a patient's body;
transecting a vessel adjacent said incision to provide a free end of said vessel;
inserting a device over said free end of said vessel, said device comprising a mechanism for self-guiding the device over said vessel; and
actuating said mechanism to advance said device over said vessel, whereby said device is self-guided over said vessel.

25. The method of claim 24, wherein said mechanism is a linear translating mechanism and said actuating comprises linearly translating said device over said vessel.

26. The method of claim 24, wherein said mechanism is automatically halted upon contact with an obstruction.

27. The method of claim 24, wherein said methods further comprises processing an obstruction contacted by said device.

28. The method of claim 27, wherein said device comprises a processing portion and said processing is performed by said processing portion.

29. The method of claim 28, wherein said processing portion comprises an inner tubular member and an outer tubular member and said processing comprises rotating at least one of said inner tubular member and said outer tubular member with respect to the other.

30. The method of claim 29, wherein said inner tubular member comprises said at least one radial extension and said outer tubular member comprises at least one groove for receiving said radial extension and said processing comprises positioning an obstruction adjacent said at least one radial extension and said at least one groove and rotating at least one of said at least one radial extension and at least one groove with respect to the other.

31. The method of claim 27, wherein said processing comprises inserting one or more tools through the lumen of said device and processing said obstruction with said one or more tools.

32. The method of claim 27, wherein said processing comprises at least one of ligating, cutting, electrocauterizing, grasping and compressing said obstruction.

33. The method of claim 24, further comprising inserting a support member in a lumen of said vessel.

34. The method of claim 33, wherein said support member is a light emitting support member and said method comprises connecting said light emitting support member to a light source.

35. The method of claim 34, wherein said support member is a light-emitting fiber optic cable or bundle thereof.

36. The method of claim 24, further comprising retaining said vessel with a lumen of said device and removing said device-retained vessel from said body.

37. A kit for harvesting a vessel from a patient's body, said kit comprising:
a device adapted to harvest vessel from a patient's body, said device comprising a mechanism for self-guiding the device over said vessel; and
a plurality of tubular body portions for use with said mechanism.

38. The kit of claim 37, further comprising a vessel support member.

39. The kit of claim 38, wherein said vessel support member is a light-emitting vessel support member.

40. The kit of claim 39, wherein said vessel support member is a light-emitting fiber optic cable or bundle thereof.
